(19) United States
(12) Patent Application Publication
(10) Pub. No.: US 2024/0376026 A1
(43) Pub. Date: Nov. 14, 2024

(54) METHODS FOR PROCESSING CHEMICALS

(71) Applicant: Dow Global Technologies LLC, Midland, MI (US)

(72) Inventors: Lin Luo, Sugarland, TX (US); Hangyao Wang, Pearland, TX (US); Yu Liu, Lake Jackson, TX (US); Matthew T. Pretz, Lake Jackson, TX (US)

(73) Assignee: Dow Global Technologies LLC, Midland, MI (US)

(21) Appl. No.: 18/695,957

(22) PCT Filed: Oct. 3, 2022
(86) PCT No.: PCT/US2022/077462

§ 371 Date: Mar. 27, 2024

Related U.S. Application Data
(60) Provisional application No. 63/251,873, filed on Oct. 4, 2021.

(51) Int. Cl. C07C 5/32; B01J 38/02

(52) U.S. Cl. CPC C07C 5/321; B01J 38/02; C07C 2601/16

(57) ABSTRACT

According to one or more embodiments of the present disclosure, a method for processing chemicals may include reacting a feed stream in the presence of a catalyst to form a product stream, passing the catalyst to a regenerator, removing olefins from a supplemental fuel stream to form an olefin depleted supplemental fuel stream, passing the olefin depleted supplemental fuel stream to the regenerator, combusting the olefin depleted supplemental fuel stream in the regenerator to heat the catalyst, and passing the heated catalyst to the reactor. The supplemental fuel stream may include at least 90 mol. % of the combination of hydrogen, methane, and nitrogen. The supplemental fuel stream may include from 0.1 mol. % to 10 mol. % olefins. The olefin depleted supplemental fuel stream may include less than or equal to 50% of the olefins present in the supplemental fuel stream prior to the olefin removal.

### CROSS-REFERENCE TO RELATED APPLICATION

This application claims the benefit of and priority to U.S. Application Ser. No. 63/251,873 filed on Oct. 4, 2021, and entitled “Methods for Processing Chemicals,” the entire contents of which are incorporated by reference in the present disclosure.

### TECHNICAL FIELD

Embodiments described herein generally relate to chemical processing and, more specifically, to methods and systems for catalytic chemical conversion.

### BACKGROUND

Chemical products may be produced by processes that employ catalysts. During these processes, the catalyst may become “spent” and have a reduced activity in subsequent reactions. Additionally, endothermic processes require heat and the “spent” catalyst may need to be reheated. Thus, spent catalyst may be transferred from a reactor to a regenerator to be reheated and regenerated, increasing the activity of the catalyst for use in further reactions. Following regeneration, the catalyst may be transferred back to the reactor for use in subsequent reactions.

### SUMMARY

Regenerating catalyst may include burning a supplemental fuel in the regenerator to heat the catalyst. The supplemental fuel may be obtained from a variety of sources, including the off-gas of a propane dehydrogenation or steam cracking process. The supplemental fuel obtained from some sources, such as the off-gas of some steam cracking processes, may include olefins. It has been discovered that the olefins found in the supplemental fuel may lead to coke formation on the fuel gas distributor in the regenerator when the regenerator is at its operating temperature. The formation of coke on the fuel gas distributor is undesirable and may lead to process interruptions. The addition of sulfur to the supplemental fuel may reduce the rate of coke formation; however, introducing sulfur into the supplemental fuel may require management of SOx formation in fuel gas and have a negative impact on the performance of the catalyst in the reactor, once the regenerated catalyst is returned to the reactor for use in further reactions.

Accordingly, there is a need for improved methods for treating fuel gas to reduce the formation of coke on the regenerator fuel gas distributor. The methods described herein address one or more of these problems. As described herein, at least a portion of the olefins contained in the supplemental fuel may be removed before the supplemental fuel is passed to the regenerator. Removing olefins from the supplemental fuel may reduce the rate of coke formation on the fuel gas distributor in the regenerator. Reducing the rate of coke formation on the fuel gas distributor may be desirable to maintain an even distribution of fuel gas throughout the regenerator.

According to one or more embodiments of the present disclosure, a method for processing chemicals may include reacting a feed stream in the presence of a catalyst in a reactor to form a product stream and passing the catalyst to a regenerator. The method may further include removing olefins from a supplemental fuel stream to form an olefin depleted supplemental fuel stream. The supplemental fuel stream comprises at least 90 mol. % of the combination of hydrogen, methane, and nitrogen. The supplemental fuel stream comprises from 0.1 mol. % to 10 mol. % olefins prior to the removal of the olefins from the supplemental fuel stream. The olefin depleted supplemental fuel stream comprises less than or equal to 50% of the olefins present in the supplemental fuel stream prior to the olefin removal. The method may further include passing the olefin depleted supplemental fuel stream to the regenerator, combusting the olefin depleted supplemental fuel stream in the regenerator to heat the catalyst to form a heated catalyst, and passing the heated catalyst to the reactor.

Additional features and advantages of the technology disclosed herein will be set forth in the detailed description which follows, and in part will be readily apparent to those skilled in the art from that description or recognized by practicing the technology as described herein, including the detailed description which follows, the claims, as well as the appended drawings.

### BRIEF DESCRIPTION OF THE DRAWINGS

The following detailed description of specific embodiments of the present disclosure can be best understood when read in conjunction with the following drawings, where like structure is indicated with like reference numerals and in which:

FIG. 1 schematically depicts a system for processing chemicals, according to one or more embodiments disclosed herein; and

FIG. 2 schematically depicts a reactor and regenerator for producing olefins, according to one or more embodiments disclosed herein.

It should be understood that the drawings are schematic in nature, and do not include some components of a fluid catalytic reactor system commonly employed in the art, such as, without limitation, temperature transmitters, pressure transmitters, flow meters, pumps, valves, and the like. It would be known that these components are within the spirit and scope of the present embodiments disclosed. However, operational components, such as those described in the present disclosure, may be added to the embodiments described in this disclosure.

Reference will now be made in greater detail to various embodiments, some embodiments of which are illustrated in the accompanying drawings. Whenever possible, the same reference numerals will be used throughout the drawings to refer to the same or similar parts.

### DETAILED DESCRIPTION

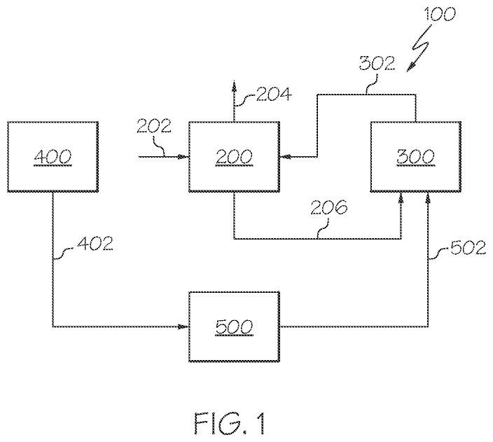

As described herein, methods for processing chemicals may include reacting a feed stream in the presence of a catalyst in a reactor to form a product stream and passing the catalyst to a regenerator. Olefins may be removed from a supplemental fuel stream to form an olefin depleted supplemental fuel stream, which may be passed to the regenerator. The olefin depleted supplemental fuel stream may be combusted in the regenerator to heat the catalyst, and the catalyst may be returned to the reactor after catalyst regeneration, which may include one or more of the removal of coke on the catalyst, heating the catalyst by combusting olefin depleted supplemental fuel, and catalyst reactivation with an oxygen treatment step. The methods described herein may be suitable for use in a system, such as the system depicted in FIG. 1. However, it should be understood that the principles disclosed and taught herein may be applicable to other systems which utilize different system components oriented in different ways.

As described herein, the term “olefins” refers to compounds made up of hydrogen and carbon that contains one or more pairs of carbon atoms linked by a double bond. For examples, olefins include ethylene, propylene, or butene. As described herein, butene many include any isomer of butene, such as 1-butene, cis-2-butene, trans-2-butene, and isobutene.

Now referring to FIG. 1, as may be understood with reference to the forgoing figures and description, in a system 100 for processing chemicals, a feed stream 202 may be reacted in a reactor 200 in the presence of a catalyst to form a product stream 204. The catalyst may be passed to the regenerator 300 by catalyst stream 206. In the regenerator 300, the catalyst may be heated and reactivated. Heating the catalyst may include combusting a supplemental fuel in the regenerator 300, in addition to combusting coke present on the catalysts in some embodiments. Supplemental fuel stream 402 from a supplemental fuel source 400 may include olefins. The olefins may be removed from the supplemental fuel stream 402 in a supplemental fuel treatment system 500 to form an olefin depleted supplemental fuel stream 502, which may be passed to the regenerator section 300. The heated and reactivated catalyst may be passed back to the reactor section 200 in stream 302 for subsequent cycles of the reaction.

The method for processing chemicals may include reacting a feed stream 202 in the presence of a catalyst in a reactor 200 to form a product stream 204. The chemical stream that is processed may be referred to as a feed stream 202, which is processed by a reaction to form a product stream 204. The feed stream 202 may comprise a composition, and depending upon that feed stream composition, an appropriate catalyst may be utilized to convert the contents of the feed stream 202 into a product stream 204. In some embodiments, the feed stream 202 may include alkanes or alkyl aromatics and the product stream 204 may include light olefins.

As described herein, a “reactor” refers to a drum, barrel, vat, or other container suitable for a given chemical reaction. A reactor may be generally cylindrical in shape (i.e., having a substantially circular diameter), or may alternately be non-cylindrically shaped, such as prism shaped with cross-sectional shaped of triangles, rectangles, pentagons, hexagons, octagons, ovals, or other polygons or curved closed shapes, or combinations thereof. Reactors, as used throughout this disclosure, may generally include a metallic frame, and may additionally include refractory linings or other materials utilized to protect the metallic frame and/or control process conditions.

The method for processing chemicals described herein may include removing olefins from a supplemental fuel stream 402 to form an olefin depleted supplemental fuel stream 502. In one or more embodiments, the supplemental fuel stream 402 may comprise one or more combustible or non-combustible gasses. For example, the supplemental fuel stream 402 may comprise hydrogen, methane, ethane, nitrogen, or combinations of these gases. In embodiments, the supplemental fuel stream 402 may comprise at least 90 mol. % of the combination of hydrogen, methane, nitrogen and ethane. For example, the supplemental fuel stream 402 may comprise at least 90 mol. %, at least 92 mol. %, at least 95 mol. %, at least 97 mol. %, at least 99 mol. % or at least 99.9 mol. % of the combination of hydrogen, methane, nitrogen and ethane. In embodiments, the supplemental fuel stream 402 comprises from 0.1 mol. % to 10 mol. % olefins. For example, the supplemental fuel stream may comprise from 0.1 mol. % to 10 mol. %, from 2 mol. % to 10 mol. %, from 4 mol. % to 10 mol. %, from 6 mol. % to 10 mol. % from 8 mol. % to 10 mol. % from 0.1 mol. % to 8 mol. %, from 0.1 mol. % to 6 mol. %, from 0.1 mol. % to 4 mol. %, from 0.1 mol. % to 2 mol. %, or any combination or sub-set of these ranges. In some embodiments, the supplemental fuel stream 402 may further comprise carbon monoxide, such as in amounts of less than 1 mol. %, less than 0.1 mol. %, or even less.

In one or more embodiments, olefins may be removed from the supplemental fuel stream 402 to form an olefin depleted supplemental fuel stream 502. Removing olefins from the supplemental fuel stream 402 may occur in olefin removal system 500. The olefin depleted supplemental fuel stream 502 may comprise less than or equal to 50 mol. % of the olefins present in the supplemental fuel stream 402 prior to the olefin removal. For example, the supplemental fuel stream 402 may comprise less than or equal to 50 mol. %, 40 mol. %, 30 mol. %, 20 mol. %, 10 mol. %, 5 mol. %, or 1 mol. % of the olefins present in the supplemental fuel stream 402 prior to the olefin removal. In embodiments, the olefin depleted supplemental fuel stream 502 may be substantially free of olefins. As described herein, a stream “substantially free” of olefins comprises less than 0.1 mol. % olefins, less than 0.05 mol. % olefins, or even less than 0.01 mol. % olefins.

In one or more embodiments, removing olefins from the supplemental fuel stream or the off-gas stream may comprise a hydrogenation reaction. As described herein, a “hydrogenation reaction” refers to a reaction in which hydrogen atoms are added to a molecule. For example, a hydrogenation reaction may be used to saturate double bonds in an alkene to form an alkane. Additionally, a hydrogenation reaction may be used to saturate triple bonds in alkynes, such as acetylene, to form alkanes. Furthermore, hydrogenation of carbon monoxide, which may be present in the supplemental fuel stream, may result in the formation of methane. In embodiments, the olefins in the supplemental fuel stream or off-gas stream may be hydrogenated to form alkanes, effectively removing the olefins from the supplemental fuel stream or off-gas stream. In such embodiments, the olefin removal system 500 may be operable to perform a hydrogenation reaction.

In one or more embodiments, the hydrogenation reaction may occur in a fixed bed reactor. As described herein, a “fixed bed reactor” is a vessel where at least a portion of the vessel is packed with a bed of catalyst such that reactants pass through the bed of catalyst and are converted to products. The fixed bed reactor may be any fixed bed reactor operable to hydrogenate olefins. In embodiments, the fixed bed reactor may be an adiabatic fixed bed reactor. In embodiments, the fixed bed reactor may be an isothermal fixed bed reactor.

The catalyst in the bed of catalyst in the fixed bed reactor may be any catalyst suitable for hydrogenating olefins. In embodiments where carbon monoxide is present in the stream, the catalyst may further be suitable for hydrogenating carbon monoxide. In embodiments, the catalyst may comprise Cu, Zn, Ni, Co, Mo, W, Pd, Rh, Pt, and combinations thereof. In embodiments, the catalyst may comprise oxides or sulfides of the metals contemplated herein. The catalyst may further comprise a support. The support may comprise one or more of an alumina, silica, zirconia, and titania. In embodiments, the catalyst may comprise a CoMoSx/NiMoSx catalyst. In embodiments, the catalyst may comprise a supported Ni catalyst. In embodiments, the catalyst may comprise a supported Pd catalyst or a supported Pd-Ag catalyst.

According to one or more embodiments, the fixed bed reactor may operate at process conditions sufficient to convert olefins in the supplemental fuel or off-gas to alkanes. In embodiments, the fixed bed reactor may operate at a temperature from 30° C. to 300° C. For example, the fixed bed reactor may operate at a temperature from 30° C. to 300° C., from 50° C. to 300° C., from 100° C. to 300° C., from 150° C. to 300° C., from 200° C. to 300° C., from 250° C. to 300° C., from 30° C. to 250° C., from 30° C. to 200° C., from 30° C. to 150° C., from 30° C. to 100° C., from 30° C. to 50° C., or any combination or sub-set of these ranges. In one or more embodiments, the fixed bed reactor may operate at a temperature suitable for the catalyst being used in the fixed bed. For example, when the catalyst comprise Ni, the temperature of the fixed bed reactor may be from 210° C. to 300° C.

In one or more embodiments, the fixed bed reactor may operate at a pressure from 25 psia to 500 psia. For example, the fixed bed reactor may operate at a pressure from 25 psia to 500 psia, from 50 psia to 500 psia, from 100 psia to 500 psia, from 150 psia to 500 psia, from 200 psia to 500 psia, from 250 psia to 500 psia, from 300 psia to 500 psia, from 350 psia to 500 psia, from 400 psia to 500 psia, from 450 psia to 500 psia, 25 psia to 450 psia, 25 psia to 400 psia, 25 psia to 350 psia, 25 psia to 300 psia, 25 psia to 250 psia, 25 psia to 200 psia, 25 psia to 150 psia, 25 psia to 100 psia, 25 psia to 50 psia, or any combination or sub-set of these ranges.

In one or more embodiments, the fixed bed reactor may have a gas hourly space velocity (GHSV) from 500 h⁻¹ to 10,000 h⁻¹. For example, the fixed bed reactor may have a GHSV from 500 h⁻¹ to 10,000 h⁻¹, 1,000 h⁻¹ to 10,000 h⁻¹, 3,000 h⁻¹ to 10,000 h⁻¹, 5,000 h⁻¹ to 10,000 h⁻¹, 7,000 h⁻¹ to 10,000 h⁻¹, 9,000 h⁻¹ to 10,000 h⁻¹, 500 h⁻¹ to 9,000 h⁻¹, 500 h⁻¹ to 7,000 h⁻¹, 500 h⁻¹ to 5,000 h⁻¹, 500 h⁻¹ to 3,000 h⁻¹, 500 h⁻¹ to 1,000 h⁻¹, or any combination or sub-set of these ranges.

In one or more embodiments, removing olefins from the supplemental fuel stream may comprise separating the olefins from the remainder of the supplemental fuel stream. In such embodiments, the olefin removal means 500 may be operable to separate olefins from the supplemental fuel stream 402. In embodiments, the separation of olefins from the supplemental fuel or off-gas stream may be achieved by membrane separation. The membrane separation process may employ a membrane to separate a permeate from a retentate, where the permeate passes through the membrane and the retentate does not pass through the membrane. In one or more embodiments, the membrane may operable to separate olefins from the alkanes and other constituents of the supplemental fuel stream. In one or more embodiments, the membrane may comprise polyimide membrane materials or polysulfone membrane materials.

In one or more embodiments, the separation of olefins from the supplemental fuel stream or off-gas stream may be achieved by an adsorption process. The adsorption process may be any adsorption process suitable for separating olefins from the paraffins or alkanes in the supplemental fuel stream or the off-gas stream. In embodiments, the adsorption process may include pressure swing adsorption, vacuum swing adsorption, or temperature swing adsorption.

The method for processing chemicals may include passing the olefin depleted supplemental fuel stream 502 to the regenerator 300. In one or more embodiments, the olefin depleted supplemental fuel stream 502 may be introduced into the regenerator 300 through one or more fuel gas distributors. Each of the one or more fuel gas distributors may comprise a plurality of fuel gas injection diffusers. The fuel gas injection diffusers permit the olefin depleted supplemental fuel stream to exit the one or more fuel gas distributors and pass into the regenerator. The one or more fuel gas distributors and fuel gas injection diffusers may be arranged to provide an even distribution of olefin depleted supplemental fuel to the regenerator. Fuel gas distributors and fuel gas injection diffusors that may be use in the regenerator 300 in one or more embodiments are described in detail in U.S. Pat. No. 9,889,418.

Without intending to be bound by theory, the presence of olefins in a supplemental fuel stream fed to the regenerator may lead to the formation of coke on the fuel gas distributors and fuel gas injection diffusers. Reducing the concentration of olefins in the supplemental fuel stream to form an olefin depleted supplemental fuel stream, and passing the olefin depleted supplemental fuel stream to the regenerator may result in reduced coke formation on the fuel gas distributors and fuel gas injection diffusers. Coke formation on the fuel gas distributors and fuel gas injection diffusers may result in uneven distribution of fuel gas throughout the regenerator. Furthermore, the removal of coke from the fuel gas distributors and fuel gas injections may result in system downtime. Minimizing the accumulation of coke on the fuel gas distributors and injectors may facilitate the even distribution of fuel gas in the regenerator 300 and reduce the need for maintenance on the fuel gas distributors and injectors.

In one or more embodiments, the temperature of the one or more fuel gas distributors in the regenerator 300 may be from 600° C. to 925° C. For example, the temperature of the one or more fuel gas distributors in the regenerator 300 may be from 600° C. to 925° C., from 600° C. to 900° C., from 600° C. to 880° C., from 600° C. to 860° C., from 600° C. to 840° C., from 600° C. to 820° C., from 600° C. to 800° C., from 600° C. to 780° C., from 600° C. to 760° C., from 600° C. to 740° C., from 600° C. to 720° C., from 600° C. to 700° C., from 600° C. to 680° C., from 600° C. to 660° C., from 600° C. to 640° C., from 600° C. to 620° C., from 620° C. to 925° C., from 640° C. to 925° C., from 660° C. to 925° C., from 680° C. to 925° C., from 700° C. to 925° C., from 720° C. to 925° C., from 740° C. to 925° C., from 760° C. to 925° C., from 780° C. to 925° C., from 800° C. to 925° C., from 820° C. to 925° C., from 840° C. to 925° C., from 860° C. to 925° C., from 880° C. to 925° C., from 900° C. to 925° C., or any combination or sub-set of these ranges. Without intending to be bound by theory, when the temperature of the one or more fuel gas distributors is from 600° C. to 780° C., coke may form on the one or more fuel gas distributors when the supplemental fuel includes olefins. Reducing the concentration of olefins in the supplemental fuel may reduce the rate of coke formation on the one or more fuel gas distributors when the fuel gas distributors are at temperatures from 600° C. to 780° C.

The method for processing chemicals may include combusting the olefin depleted supplemental fuel stream 502 in the regenerator 300 to heat the catalyst to form a heated catalyst. In one or more embodiments, the temperature of the heated catalyst is greater than the temperature of the catalyst passed to the regenerator in stream 206. The heated catalyst may be passed from the regenerator 300 to the reactor 200 in stream 302. In one or more embodiments, the catalyst may be heated in the regenerator 300 to a temperature sufficient to maintain the heat balance of the reactor 300. In other words, the catalyst, heated in the regenerator 300, may be the primary source of heat used to maintain the temperature of the reactor 200.

In one or more embodiments the heated catalyst may be further treated by contacting the heated catalyst with oxygen to form an oxygen-treated catalyst, and the oxygen-treated catalyst may be passed to the reactor. For example, the heated catalyst may be contacted with an oxygen containing gas, such as air, enriched air, or even pure oxygen. The oxygen-treated catalyst may have increased activity for one or more reactions occurring within the reactor, including but not limited to dehydrogenation reactions.

In one or more embodiments, the supplemental fuel stream 402 may be an off-gas from a dehydrogenation process or a steam cracking process. For example, the supplemental fuel stream 402 may be an off-gas from a propane dehydrogenation process, an ethylbenzene dehydrogenation process, a butane dehydrogenation process, an ethane dehydrogenation process, or a steam cracking process.

In one or more embodiments, the supplemental fuel stream 402 is an off-gas from a steam cracking process. In such embodiments, the fuel gas source 400 of FIG. 1 is a steam cracking system. The steam cracking system may be operable to produce an off-gas stream, which may be used as a supplemental fuel stream, and a steam cracking product stream from a hydrocarbon feed.

In one or more embodiments, steam cracking a hydrocarbon feed may occur in a steam cracking unit. The steam cracking unit may be operable to receive the hydrocarbon feed and crack one or more constituents of the hydrocarbon feed to form at least an off-gas stream and a steam cracking product stream. Ethane, propane, naphtha, and other hydrocarbons present in the hydrocarbon feed may be steam cracked in the steam cracking unit to produce at least one or more olefins, such as but not limited to ethylene, propylene, butenes, or combinations of these. The steam cracking unit may be operated under conditions (i.e., temperature, pressure, residence time, etc.) sufficient to produce one or more light olefins such as ethylene and propylene from the hydrocarbons in the hydrocarbon feed. In some embodiments, the steam cracking unit may be operated at a temperature of from 500° C., to 950° C., from 500° C. to 900° C., from 600° C. to 950° C., from 600° C. to 900° C., from 700° C. to 950° C., or from 700° C. to 900° C. The temperature of the steam cracking unit may depend on the composition of the hydrocarbon feed introduced to the steam cracking unit.

The hydrocarbon feed may be any hydrocarbon stream, such as a product stream from a petrochemical process or naphtha from a refining operation for crude oil, natural gas liquids (NGL), or other hydrocarbon sources. In some embodiments, the hydrocarbon feed may include a plurality of different hydrocarbon streams combined prior to or in the steam cracking unit. In some embodiments, the hydrocarbon feed may be a light hydrocarbon feedstock, such as a feedstock including ethane, propane, butane, naphtha, other light hydrocarbon, or combinations of these.

In one or more embodiments, the steam cracking product stream may include one or more cracking reaction products, such as, but not limited to ethylene, propylene, butenes (e.g., 1-butene, trans-2-butene, cis-2-butene, isobutene) or combinations of these.

The off-gas stream may comprise at least 90 mol. % of the combination of hydrogen, methane, and nitrogen. For example, the off-gas stream may comprise at least 90 mol. %, at least 92 mol. %, at least 95 mol. %, at least 97 mol. %, at least 99 mol. % or at least 99.9 mol. % of the combination of hydrogen, methane, and nitrogen. The off-gas stream may comprise from 0.1 mol. % to 10 mol. % olefins. For example, the off-gas stream may comprise from 0.1 mol. % to 10 mol. %, from 2 mol. % to 10 mol. %, from 4 mol. % to 10 mol. %, from 6 mol. % to 10 mol. % from 8 mol. % to 10 mol. % from 0.1 mol. % to 8 mol. %, from 0.1 mol. % to 6 mol. %, from 0.1 mol. % to 4 mol. %, from 0.1 mol. % to 2 mol. %, or any combination or sub-set of these ranges. In one or more embodiments, at least a portion of the off-gas stream may be the supplemental fuel stream 402.

In one or more embodiments, the reaction occurring in reactor 200 may be a dehydrogenation reaction. The dehydrogenation reaction may be a thermal dehydrogenation reaction or a catalytic dehydrogenation reaction. According to such embodiments, the feed stream 202 may comprise one or more of ethylbenzene, ethane, propane, n-butane, and i-butane. In one or more embodiments, the feed stream may 202 comprise at least 50 wt. %, at least 60 wt. %, at least 70 wt. %, at least 80 wt. %, at least 90 wt. %, at least 95 wt. % or even at least 99 wt. % of ethane. In additional embodiments, the feed stream 202 may comprise at least 50 wt. %, at least 60 wt. %, at least 70 wt. %, at least 80 wt. %, at least 90 wt. %, at least 95 wt. % or even at least 99 wt. % of propane. In additional embodiments, the feed stream 202 may comprise at least 50 wt. %, at least 60 wt. %, at least 70 wt. %, at least 80 wt. %, at least 90 wt. %, at least 95 wt. % or even at least 99 wt. % of n-butane. In additional embodiments, the feed stream 202 may comprise at least 50 wt. %, at least 60 wt. %, at least 70 wt. %, at least 80 wt. %, at least 90 wt. %, at least 95 wt. % or even at least 99 wt. % of i-butane. In additional embodiments, the feed stream 202 may comprise at least 50 wt. %, at least 60 wt. %, at least 70 wt. %, at least 80 wt. %, at least 90 wt. %, at least 95 wt. % or even at least 99 wt. % of the sum of ethane, propane, n-butane, and i-butane.

In one or more embodiments, the product stream 204 may comprise at least 30 wt. % olefins. For example, the product stream 204 may comprise at least 30 wt. % olefins, at least 40 wt. % olefins, at least 50 wt. % olefins, or even at least 60 wt. % olefins. In one or more embodiments, the olefins comprising the product stream may comprise one or more of ethylene, propylene, styrene, and butenes, such as 1-butene, trans-2-butene, cis-2-butene, and isobutene.

In one or more embodiments, the dehydrogenation reaction may utilize gallium and/or platinum particulate solids as a catalyst. In such embodiments, the catalyst may comprise a gallium and/or platinum catalyst. As described herein, a gallium and/or platinum catalyst comprises gallium, platinum, or both. The gallium and/or platinum catalyst may be carried by an alumina or alumina silica support, and may optionally comprise potassium. Such gallium and/or platinum catalysts are disclosed in U.S. Pat. No. 8,669,406, which is incorporated herein by reference in its entirety. However, it should be understood that other suitable catalysts may be utilized to perform the dehydrogenation reaction. For example, in embodiments, mixed metal oxides may be suitable catalysts for performing the dehydrogenation reaction. In one or more embodiments, the catalyst may comprise a combination of multiple catalysts, such as but not limited to, a mixed metal oxide catalyst and a gallium and/or platinum catalyst.

In one or more embodiments, the catalyst may comprise Geldart A particles. Geldart A particles generally exhibit a small mean particle size and/or low particle density (<˜1.4 grams per cubic centimeter, g/cm³), fluidize easily with smooth fluidization at low gas velocities, and exhibit controlled bubbling with small bubbles at higher gas velocities. In one or more embodiments, Geldart A particles may form an aeratable powder, having a bubble-free range of fluidization; a high bed expansion; a slow and linear deaeration rate; bubble properties that include a predominance of splitting/recoalescing bubbles, with a maximum bubble size and large wake; high levels of solids mixing and gas backmixing, assuming equal U−U_{mf} (U is the velocity of the carrier gas, and U_{mf} is the minimum fluidization velocity, typically though not necessarily measured in meters per second, m/s, i.e., there is excess gas velocity); axisymmetric slug properties; and no spouting, except in very shallow beds. The properties listed tend to improve as the mean particle size decreases, assuming equal [symbol] ; or as the <45 micrometers (μm) proportion is increased; or as pressure, temperature, viscosity, and density of the gas increase.

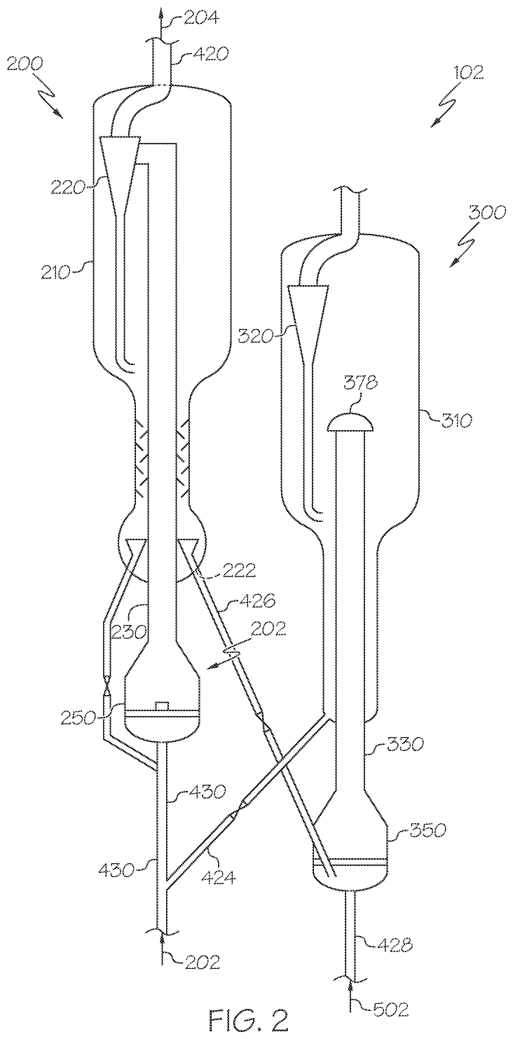

In one or more embodiments, the reactor 200 and regenerator 300 of FIG. 1 may be configured as depicted in FIG. 2. However, it should be understood that other reactor system configurations may be suitable for the methods described herein. Now referring to FIG. 2, an example reactor system 102 which may be suitable for use with the methods described herein is schematically depicted. The reactor system 102 generally comprises multiple system components, such as a reactor 200 and/or a regenerator 300. As used herein in the context of FIG. 1, the reactor 200 generally refers to the portion of a reactor system 102 in which the major process reaction takes place. The reactor 200 comprises a reactor vessel 202 which may include a downstream reactor section 230 and an upstream reactor section 250. According to one or more embodiments, as depicted in FIG. 2, the reactor 200 may additionally include a catalyst separation section 210, which serves to separate the catalyst from the chemical products formed in the reactor vessel 202. Also, as used herein, the regenerator 300 generally refers to the portion of a reactor system 102 where the catalyst is in some way processed, such as by combustion. The regenerator portion 300 may comprise a combustor 350 and a riser 330, and may optionally comprise a catalyst separation section 310. In some embodiments, the catalyst may be regenerated by burning off contaminants like coke in the regenerator 300. In embodiments, the catalyst may be heated in the regenerator 300. An olefin depleted supplemental fuel may be utilized to heat the catalyst in the regenerator 300. In one or more embodiments, the catalyst separation section 210 may be in fluid communication with the combustor 350 (e.g., via standpipe 426) and the catalyst separation section 310 may be in fluid communication with the upstream reactor section 250 (e.g., via standpipe 424 and transport riser 430).

As described with respect to FIG. 2, the feed stream 202 may enter transport riser 430, and the product stream 204 may exit the reactor system 102 via pipe 420. According to one or more embodiments, the reactor system 102 may be operated by feeding a chemical feed (e.g., in a feed stream) and a fluidized catalyst into the upstream reactor section 250. The chemical feed contacts the catalyst in the upstream reactor section 250, and each flow upwardly into and through the downstream reactor section 230 to produce a chemical product. The chemical product and the catalyst may be passed out of the downstream reactor section 230 to a separation device 220 in the catalyst separation section 210, where the catalyst is separated from the chemical product, which is transported out of the catalyst separation section 210. The separated catalyst is passed from the catalyst separation section 210 to the combustor 350. In the combustor 350, the catalyst may be processed by, for example, combustion. For example, and without limitation, the catalyst may be de-coked and olefin depleted supplemental fuel may be combusted to heat the catalyst. The olefin depleted supplemental fuel 502 may be passed to the combustor 350 through pipe 428. The catalyst is then passed out of the combustor 350 and through the riser 330 to a riser termination separator 378, where the gas and solid components from the riser 330 are at least partially separated. The vapor and remaining solids are transported to a secondary separation device 320 in the catalyst separation section 310 where the remaining catalyst is separated from the gases from the catalyst processing (e.g., gases emitted by combustion of spent catalyst or supplemental fuel). The separated catalyst is then passed from the catalyst separation section 310 to the upstream reactor section 250 via standpipe 424 and transport riser 430, where it is further utilized in a catalytic reaction. Thus, the catalyst, in operation, may cycle between the reactor portion 200 and the catalyst processing portion 300. In general, the processed chemical streams, including the feed streams and product streams may be gaseous, and the catalyst may be fluidized particulate solid.

It should be understood that any two quantitative values assigned to a property may constitute a range of that property, and all combinations of ranges formed from all stated quantitative values of a given property are contemplated in this disclosure. It should be appreciated that compositional ranges of a chemical constituent in a composition should be appreciated as containing, in some embodiments, a mixture of isomers of that constituent. In additional embodiments, the chemical compounds may be present in alternative forms such as derivatives, salts, hydroxides, etc. Generally, “inlet ports” and “outlet ports” of any system unit of the reactor system 102 described herein refer to openings, holes, channels, apertures, gaps, or other like mechanical features in the system unit. For example, inlet ports allow for the entrance of materials to the particular system unit and outlet ports allow for the exit of materials from the particular system unit. Generally, an outlet port or inlet port will define the area of a system unit of the reactor system 102 to which a pipe, conduit, tube, hose, transport line, or like mechanical feature is attached, or to a portion of the system unit to which another system unit is directly attached. While inlet ports and outlet ports may sometimes be described herein functionally in operation, they may have similar or identical physical characteristics, and their respective functions in an operational system should not be construed as limiting on their physical structures.

### EXAMPLES

The following examples illustrate features of the present disclosure but are not intended to limit the scope of the disclosure. The following examples discuss the rate of coke formation on stainless steel, according to one or more embodiments disclosed herein.

The rate of coke formation on stainless steel was analyzed. Samples of an off-gas from a steam cracking process comprising 2 mol. % ethylene, 80 mol. % H₂, and 18 mol. % methane was passed through a 40 inch long 304H stainless steel tube. The stainless steel tube was coiled inside a furnace and the furnace was heated to 700° C. The off-gas was continuously fed through the stainless steel tube for the duration of the coking process, which ranges from 1 hours to 150 hours. Afterwards, the coke was burned off using a gas comprising 5 mol. % oxygen and 95 mol. % nitrogen in a decoking step. The gas produced during the decoking step was analyzed by mass spectrometry to determine the concentrations of CO and CO₂ in the gas produced during the decoking step. The concentrations of CO and CO₂ were used to determine the amount of coke that had been formed in the stainless steel tube. Then, the coke formation rate was calculated using the amount of coke, the internal surface area of the stainless steel tube, and the duration of the coking process.

The coke formation rate at 700° C. for off gas comprising 2 mol. % ethylene, 80 mol. % H₂, and 18 mol. % methane was about 3 mg/in²/hr. Assuming a constant coke growth rate and an estimated coke density 0.2 g/cm³, the thickness of the coke accumulating on system components would be about 20.4 cm/year. Coke accumulation at this rate in various system components would likely lead to disruptions in operation.

It is expected that there is no coke formation when the off gas includes only hydrogen and methane, when the concentration of ethylene is 0 mol. %. Specifically, hydrogen does not include carbon, so it cannot form coke. Additionally, the thermal decomposition of methane at 700° C. is negligible. As thermal decomposition generally leads to the formation of coke, it is expected that coke formation from methane at 700° C. would be negligible. Therefore, reducing the concentration of ethylene in the off gas should lead to a reduction in the coke formation rate.

It is noted that one or more of the following claims utilize the term “wherein” as a transitional phrase. For the purposes of defining the present technology, it is noted that this term is introduced in the claims as an open-ended transitional phrase that is used to introduce a recitation of a series of characteristics of the structure and should be interpreted in like manner as the more commonly used open-ended preamble term “comprising.”

It should be understood that where a first component is described as “comprising” a second component, it is contemplated that, in some embodiments, the first component “consists” or “consists essentially of” that second component. It should further be understood that where a first component is described as “comprising” a second component, it is contemplated that, in some embodiments, the first component comprises at least 10%, at least 20%, at least 30%, at least 40%, at least 50%, at least 60%, at least 70%, at least 80%, at least 90%, at least 95%, or even at least 99% that second component (where % can be weight % or molar %).

Additionally, the term “consisting essentially of” is used in this disclosure to refer to quantitative values that do not materially affect the basic and novel characteristic(s) of the disclosure. For example, a chemical composition “consisting essentially” of a particular chemical constituent or group of chemical constituents should be understood to mean that the composition includes at least about 99.5% of a that particular chemical constituent or group of chemical constituents.

The subject matter of the present disclosure has been described in detail and by reference to specific embodiments. It should be understood that any detailed description of a component or feature of an embodiment does not necessarily imply that the component or feature is essential to the particular embodiment or to any other embodiment. Further, it should be apparent to those skilled in the art that various modifications and variations can be made to the described embodiments without departing from the spirit and scope of the claimed subject matter.

1. A method for processing chemicals, the method comprising:
reacting a feed stream in the presence of a catalyst in a reactor to form a product stream;
passing the catalyst to a regenerator;
removing olefins from a supplemental fuel stream to form an olefin depleted supplemental fuel stream, wherein:
the supplemental fuel stream comprises at least 90 mol. % of the combination of hydrogen, methane, and nitrogen;
the supplemental fuel stream comprises from 0.1 mol. % to 10 mol. % olefins prior to the removal of the olefins from the supplemental fuel stream; and
the olefin depleted supplemental fuel stream comprises less than or equal to 50% of the olefins present in the supplemental fuel stream prior to the olefin removal;
passing the olefin depleted supplemental fuel stream to the regenerator;
combusting the olefin depleted supplemental fuel stream in the regenerator to heat the catalyst to form a heated catalyst; and
passing the heated catalyst to the reactor.

2. The method of claim 1, wherein the supplemental fuel stream is an off-gas stream from a propane dehydrogenation process, an ethylbenzene dehydrogenation process, a butane dehydrogenation process, an ethane dehydrogenation process, or a stream cracking process.

3. The method of claim 1, wherein removing olefins from the supplemental fuel stream comprises a hydrogenation reaction or separation by membrane or adsorption.

4. The method of claim 1, wherein removing olefins from the supplemental fuel stream comprises a hydrogenation reaction occurring in a fixed bed reactor.

5. The method of claim 4, wherein the fixed bed reactor operates at a temperature from 30° C. to 300° C.

6. The method of claim 4, wherein the fixed bed reactor operates at a pressure from 25 psia to 500 psia.

7. The method of claim 4, wherein the fixed bed reactor operates at a gas hourly space velocity from 500 h⁻¹ to 10,000 h⁻¹.

8. The method of claim 1, wherein reacting the feed stream comprises performing a dehydrogenation reaction.

9. The method of claim 1, wherein the feed stream comprises one or more alkanes or alkyl aromatics.

10. The method of claim 1, wherein the catalyst comprises Geldart A particles.

11. The method of claim 1, wherein the product stream comprises one or more of ethylene, propylene, butene, or styrene.

12. The method of claim 1, further comprising:
contacting the heated catalyst with oxygen to form an oxygen-treated catalyst; and
passing the oxygen-treated catalyst to the reactor.

13. The method of claim 1, wherein the method further comprises,
processing a hydrocarbon feed to form at least the supplemental fuel stream and a product stream, wherein the processing comprises one or more of propane dehydrogenation, ethylbenzene dehydrogenation, butane dehydrogenation, ethane dehydrogenation, or stream cracking; and
hydrogenating the supplemental fuel stream to form an olefin depleted supplemental fuel stream.

14. The method of claim 1, wherein the olefin depleted supplemental fuel stream is passed to the regenerator through one or more fuel gas distributors.
